(19) United States
(12) Patent Application Publication
(10) Pub. No.: US 2006/0282049 A1
(43) Pub. Date: Dec. 14, 2006

(54) Eye fluid dispensing apparatus

(76) Inventor: Don W. Lee, Glendale, CA (US)

Correspondence Address: JAMES E. BRUNTON, ESQ., P. O. BOX 29000, GLENDALE, CA, 91209, US

(21) Appl. No.: 11/150,077

(22) Filed: Jun. 11, 2005

(51) Int. Cl. A61M 35/00

(52) U.S. Cl. 604/300; 604/301

(57) ABSTRACT

An apparatus for controllably dispensing fluid, such as eyewash, from a fluid container into the human eye. The apparatus includes a uniquely formed eyecup and a connector connected to the eyecup for securely interconnecting a bottle or vial containing the treating liquid with the eyecup. The connector sealably positions the tip of the liquid containing bottle or vial within the eyecup and at a strategic spaced-apart location relative to the eye being treated.

### BACKGROUND OF THE INVENTION

1. Field of the Invention

The present invention relates generally to apparatus for dispensing medicinal fluids. More particularly, the invention concerns an eye fluid dispensing apparatus for controllably dispensing fluid, such as eyewash, from a fluid container into the human eye.

2. Discussion of the Prior Art

Various designs of eyecups have been suggested in the past and have been widely used for washing and treating the human eye with fluids such as eyewash. The conventional eyecup typically comprises an oval cup with a rim curved to fit the orbit of the eye and is used for applying liquid remedies to the eyes.

Exemplary of currently commercially available eyecups is a molded plastic eyecup sold along with an eyewash solution by Bausch & Lomb, Incorporated under the name and style COLLYRIUM. In using the eyecup, the cup portion is filled with the eye wash solution and the eyecup is then fitted over the orbit of the eye.

In the past, attempts have been made to modify a conventional eyecup to accept an eye drop bottle or the like. Such attempts include drilling a hole and the base of the eyecup of a size sufficient to accept the dispensing tip of the eye drop bottle so that the fluid contained within the eye drop bottle can be dispensed into the interior of the eyecup. More sophisticated attempts have included the provision of some type of apparatus which includes a connector to enable the interconnection of the eye drop bottle with the eyecup. Exemplary of this latter type of apparatus is an apparatus sold by the Life With Ease Company of Newbury, N.H., under the name and style “Autodrop Eye Drop Guide”. This apparatus comprises a hollow eye drop guide which is shaped to fit the contours of the face around the eye and a clip arrangement which enables a drug bottle to be clipped into place over the eye drop guide.

Many of the prior art devices which attempt to interconnect an eye drop bottle with an eyecup are quite cumbersome and awkward to use. Further, these devices typically imprecisely position the dispensing tip of the eye drop bottle relative to the eye. It is these prior art deficiencies that the apparatus of the present invention seeks to overcome by providing an apparatus that includes a uniquely configured eyecup and a novel connection means that provides a positive interconnection between the eyecup and the liquid containing bottle. Additionally, the connection means of the invention is designed to position the dispensing tip of the liquid containing bottle at a strategic location within the eyecup and relative to the eye being treated.

### SUMMARY OF THE INVENTION

It is an object of the present invention to provide an easy to use apparatus for controllably dispensing fluid, such as eyewash, from a fluid container into the human eye.

Another object of the invention is to provide an apparatus of the aforementioned character which includes a uniquely formed eyecup and connector means connected to the eyecup for securely interconnecting a bottle or vial containing the treating liquid with the eyecup.

Another object of the invention is to provide an apparatus as described in the preceding paragraph in which the connector means strategically, sealably positions the tip of the liquid containing bottle or vial within the eyecup and at a strategic spaced-apart location relative to the eye being treated.

Another object of the invention is to provide an apparatus of the class described which is of a simple design and one which is usable with most standard eye treatment liquid containing bottles and vials.

Another object to the invention is to provide an apparatus of the character described herein which is highly reliable in use and one which can be manufactured relatively inexpensively.

The foregoing, as well as other objectives, can be achieved by an apparatus, the character of which is described in the paragraphs which follow.

### BRIEF DESCRIPTION OF THE DRAWINGS

FIG. 1 is a generally perspective view of one form of the apparatus of the invention illustrating the manner in which it is used with a conventional liquid dispensing bottle.

FIG. 2 is a cross-sectional view taken along lines 2-2 of FIG. 1.

FIG. 3 is a view taken along lines 3-3 of FIG. 2.

FIG. 4 is a view taken along lines 4-4 of FIG. 2.

### DESCRIPTION OF THE INVENTION

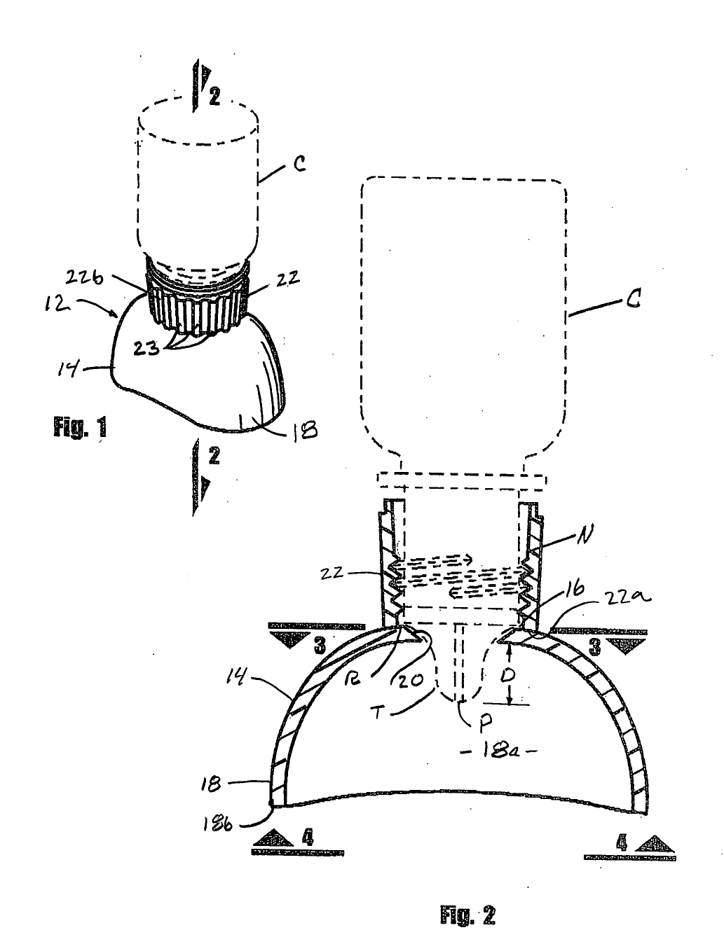

Referring to the drawings and particularly to FIGS. 1, 2 and 3, one form of the fluid dispensing apparatus of the invention for dispensing fluid from a fluid container is there shown in generally designated by the numeral 12. As indicated in FIGS. 1 and 2, the fluid container “C” that is usable with the dispensing apparatus of the invention has an externally threaded neck portion “N” and an interconnected, tapered dispensing tip “T” for controllably directing fluid from the fluid container toward the eye. Container “C”, which is normally closed by an internally threaded cap (not shown), can contain various eye treatment fluids, such as eyewash, dexamethasone ophthalmic suspension and like medicaments.

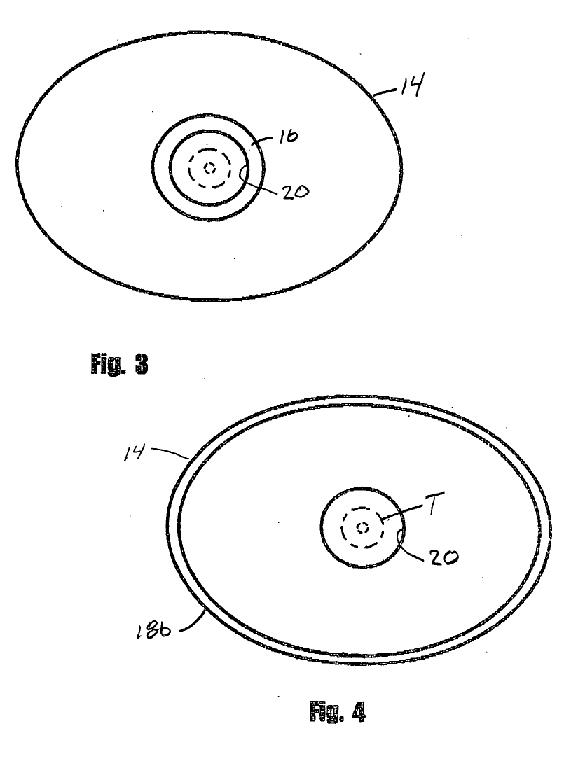

In the present form of the invention, the fluid dispensing apparatus comprises an eyecup component 14 having a generally planar, substantially circular shaped central portion 16 (FIG. 3) and a skirt portion 18. Skirt portion 18 extends outwardly and downwardly from central portion 16 in the manner best seen in FIG. 2 to define an interior space 18a. While skirt portion 18 may be of various configurations, in the present form of the invention skirt portion 18 terminates in a generally elliptically-shaped periphery 18b (FIG. 4). Eyecup component 14 can be constructed of a wide variety of materials, but is preferably constructed from a yieldably deformable, moldable plastic material.

As illustrated in FIG. 2, central portion 16 has a tapered aperture 20 for closely receiving the tapered dispensing tip “T” of the fluid container in a manner to permit the dispensing tip to extend into interior space 18a a predetermined distance “D” (see FIG. 2).

Securely interconnected to eyecup 14 is an internally threaded, generally cylindrically-shaped connector 22. As best seen in FIG. 2, connector 22 has a generally anular-shaped base periphery 22a, which is connected to central portion 16 of the eyecup as, for example, by adhesive bonding. To assist in the interconnection of the fluid container with the apparatus of the invention, the threaded connector 22 also includes an exterior surface 22b, which is provided with a plurality of gripping striations 23.

As indicated by the dotted lines in FIG. 2, threaded connector 22 functions to threadably receive the externally threaded neck portion “N” of the fluid container. When the threaded neck portion of the fluid container is threadably received within the threaded connector 22 in the manner shown in FIG. 2, the annular-shaped rim “R” of the dispensing tip of the fluid container sealably engages the generally planar central portion 16 of the eyecup.

In using the apparatus of the invention, the top of the fluid container containing the eye treatment fluid is first removed so that the threaded neck of the container “C” can be threadably interconnected with the connector portion 22 of the apparatus in the manner shown in FIGS. 1 and 2. As the neck of the container is seated against the generally planar, central top portion 16 of the eyecup, the annular-shaped rim “R” of the container neck (FIG. 2) will sealably engage the central portion 16 of the eyecup so as to substantially prevent leakage of fluid into the threaded connector portion 22 during the fluid dispensing operation. With the threaded neck of the dispensing container seated in the manner shown in FIG. 2 the dispensing tip “T” will extend into interior space 18a a predetermined, limited distance “D”. As indicated in FIG. 2 of the drawings, in the seated position, the fluid outlet passageway “P” of the dispensing tip will also be precisely centrally positioned relative to the eyecup to enable the fluid contained within the fluid container to be precisely and controllably dispensed to the patient's eye in a repeatable fashion.

With the flow dispensing container interconnected with the apparatus of the present invention in the manner shown in FIG. 2, the eyecup component 14 of the apparatus can be conveniently positioned over the patient's eye in a conventional manner to permit the controlled dispensing of fluid from the fluid container “C” into the patient's eye.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

I claim:

1. A fluid dispensing apparatus for dispensing fluid from a fluid container having a threaded neck portion and an interconnected dispensing tip for controllably directing fluid toward the eye, said apparatus comprising:
(a) an eyecup having a generally planar central portion and a skirt portion extending outwardly and downwardly from said central portion, said central portion having an aperture for closely receiving the dispensing tip of the fluid container in a manner to permit the dispensing tip to extend into said skirt portion; and
(b) a threaded connector connected to said central portion of said eyecup for threadably receiving the threaded neck portion of the fluid container.

2. The fluid dispensing apparatus as defined in claim 1 in which said skirt portion terminates in a generally elliptically-shaped periphery.

3. The fluid dispensing apparatus as defined in claim 1 in which said threaded neck portion of the fluid container sealably engages said central portion of said eyecup when said threaded neck portion is threadably received within said threaded connector.

4. The fluid dispensing apparatus as defined in claim 1 in which said eyecup is constructed from a yieldably deformable plastic material.

5. The fluid dispensing apparatus as defined in claim 1 in which said threaded connector has a generally anular-shaped base periphery connected to said central portion of said eyecup for sealably interconnecting the neck portion of the fluid container with said eyecup.

6. A fluid dispensing apparatus for dispensing fluid from a fluid container having a threaded neck portion and an interconnected dispensing tip for controllably directing fluid toward the eye, said apparatus comprising:
(a) an eyecup having a generally planar central portion and a skirt portion extending outwardly and downwardly from said central portion and terminating in a generally elliptically-shaped periphery, said central portion having an aperture for closely receiving the dispensing tip of the fluid container in a manner to permit the dispensing tip to extend into said skirt portion a predetermined distance; and
(b) a threaded connector having a generally anular-shaped base periphery connected to said central portion of said eyecup for sealably interconnecting the neck portion of the fluid container with said generally planar central portion of said eyecup said threaded connector having internal threads for threadably receiving the threaded neck portion of the fluid container.

7. The fluid dispensing apparatus as defined in claim 6 in which said eyecup is constructed from a yieldably deformable plastic material.

8. The flow dispensing apparatus as defined in claim 7 in which said threaded connector includes an exterior surface, said exterior surface being provided with a plurality of gripping striations.
